(19) United States
(12) Patent Application Publication
(10) Pub. No.: US 2023/0080468 A1
(43) Pub. Date: Mar. 16, 2023

(54) FLUIDLY-OPERATED FASTENER ASSEMBLY

(71) Applicant: Step Surgical Solutions PTE Ltd., Singapore (IL)

(72) Inventors: Yosef Hazan, Haifa (IL); Nir Goldenberg, Haifa (IL); Dotan Tromer, Moshav Hosen (IL); Avshalom Shenhav, Haifa (IL); Arnon Hadas, Moshav Avigdor (IL); Yaakov Greenberg, Even Yehuda (IL); Tiong Han Toh, Singapore (SG)

(73) Assignee: Step Surgical Solutions PTE Ltd., Singapore (SG)

(21) Appl. No.: 17/799,678

(22) PCT Filed: Feb. 17, 2021
(86) PCT No.: PCT/IB2021/051346

§ 371 Date: Aug. 14, 2022

Related U.S. Application Data
(60) Provisional application No. 62/978,896, filed on Feb. 20, 2020.

(51) Int. Cl. A61B 17/072; A61B 17/064

(52) U.S. Cl. CPC A61B 17/072; A61B 17/0644; A61B 2017/07278; A61B 2017/07228; A61B 2017/07285; A61B 2017/07257; A61B 2017/0647; A61B 2017/00557

(57) ABSTRACT

A surgical fastener assembly includes one or more arrays of fasteners, each of the fasteners including a head portion opposite to a sharp portion. Each of the head portions of the fasteners is held by a fastener head holding member. A receiver member is aligned with each of the one or more arrays of fasteners. A fluid actuator is provided for each of the one or more arrays of fasteners, and it is contractible and expandable. Each of the fluid actuators is configured to provide a force against its corresponding array of fasteners to propel the sharp portions of the fasteners towards the receiver member and also configured to provide a force to free the head portions from the fastener head holding member.

### FIELD OF THE INVENTION

The present invention generally relates to surgical tackers, and more particularly to a fluidly-operated fastener (e.g., tacker) and cutting instrument.

### BACKGROUND OF THE INVENTION

Surgical linear staplers are used to simultaneously make a longitudinal incision in tissue and apply rows of staples on opposing sides of the incision. Linear staplers include a pair of cooperating jaw members that, if the instrument is intended for endoscopic or laparoscopic applications, are capable of passing through a cannula passageway. One of the jaw members receives a staple cartridge having at least two laterally spaced rows of staples. The other jaw member defines an anvil having staple-forming pockets aligned with the rows of staples in the cartridge. Prior art staplers include reciprocating wedges that, when driven distally, pass through openings in the staple cartridge and engage drivers supporting the staples to effect the firing of the staples toward the anvil.

### SUMMARY OF THE INVENTION

The present invention seeks to provide a novel fluidly-operated tacker and cutting instrument, as described in detail below. “Fluidly-operated” means the tacker may be hydraulically or pneumatically operated. The term “tacker” encompasses a tack, a staple or any other fastener for joining tissues together. Accordingly, the terms tacker and stapler are used interchangeably.

The tacker is suitable for endoscopic or laparoscopic applications, and can be introduced through a trocar or similar device, such as through a working lumen, having a diameter, without limitation, of 5, 8, 10, 12 or 15 mm.

The tacker cartridge can be used with a great range of articulation, such as ±90°. The fluidic actuation provides a large clamping range for a single-size cartridge and can reduce operator mistakes which could occur due to improper cartridge selection.

### BRIEF DESCRIPTION OF THE DRAWINGS

The present invention will be understood and appreciated more fully from the following detailed description, taken in conjunction with the drawings in which:

FIGS. 1 and 2 are simplified exploded and cutaway illustrations of a surgical tacker assembly, showing a cartridge with fluid actuators for firing one or more rows of tacks and for actuating a cutter, in accordance with a non-limiting embodiment of the present invention;

FIG. 3 is a simplified pictorial illustration of the cartridge of the assembly (the cartridge can be reloadable);

FIGS. 4A and 4B are simplified pictorial illustrations of another cutter that can be used in the tacker assembly of the invention, respectively in a closed configuration after cutting and an open configuration prior to cutting;

FIG. 5 is a simplified pictorial illustration of a receiver (or anvil) member formed with apertures which will receive the tacks;

FIG. 6 is a simplified pictorial illustration of a row of tacks supported by an expandable support member;

FIG. 7 is a simplified pictorial illustration of a row of tacks that have been fired and are received through the apertures of the receiver member;

FIG. 8 is a simplified pictorial illustration of tissue stapled with tacks that were fired by the tacker assembly of the invention;

FIG. 9 is a simplified pictorial illustration of a surgical tacker instrument, which uses the surgical tacker assembly of any of the embodiments shown herein, including a cartridge jaw for receiving the cartridge, an anvil jaw for receiving the anvil, and controls for articulation and rotation of the jaws;

FIG. 10A is a simplified block diagram of the controls for operating the surgical tacker instrument of FIG. 9, including fluid-operated cutting, tacker firing and jaw closure and opening;

FIG. 10B is a simplified block diagram of the controls for operating the surgical tacker instrument of FIG. 9, including fluid-operated tacker firing and jaw closure and opening, and non-fluid-operated cutting;

FIG. 11A is a simplified pictorial illustration of a tack cartridge of the surgical tacker assembly for use with the instrument of FIG. 9, including a clamp balloon that provides a distributed force to clamp tissue before tacking and cutting, and a tack balloon that provides a force to fire (eject) the tacks from the cartridge;

FIG. 11B is a simplified cutaway illustration of the tack cartridge of FIG. 11A, showing deployment (expansion) of the clamp balloon;

FIG. 11C is a simplified cutaway illustration of the tack cartridge showing deployment (expansion) of the tack balloon;

FIGS. 11D and 11E are simplified end-view and end-view perspective illustrations of FIG. 11C;

FIGS. 12A and 12B are simplified pictorial illustrations of tack holders of the tack cartridge, with and without tacks, respectively;

FIGS. 13A and 13B are simplified pictorial illustrations, in different views, of the tack holders, showing two different guides to stabilize the tack holders in yaw and pitch;

FIG. 14A is a simplified illustration of non-parallel tack lines (rows) in the tacker assembly; and

FIGS. 14B and 14C are simplified pictorial illustrations using the non-parallel tack lines in tissue.

### DETAILED DESCRIPTION OF EMBODIMENTS

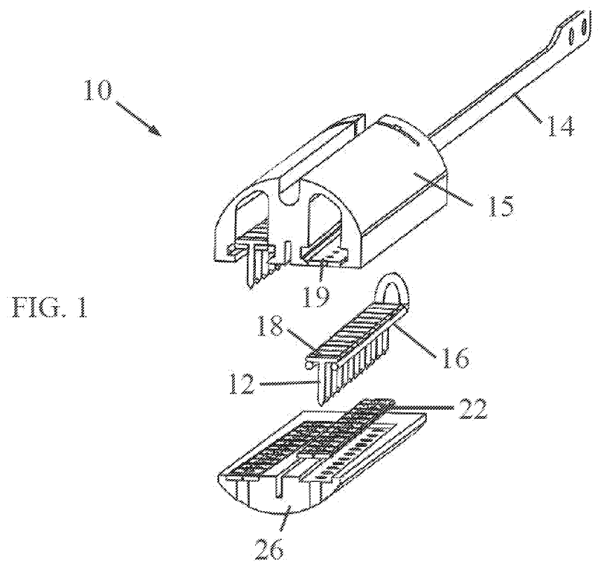

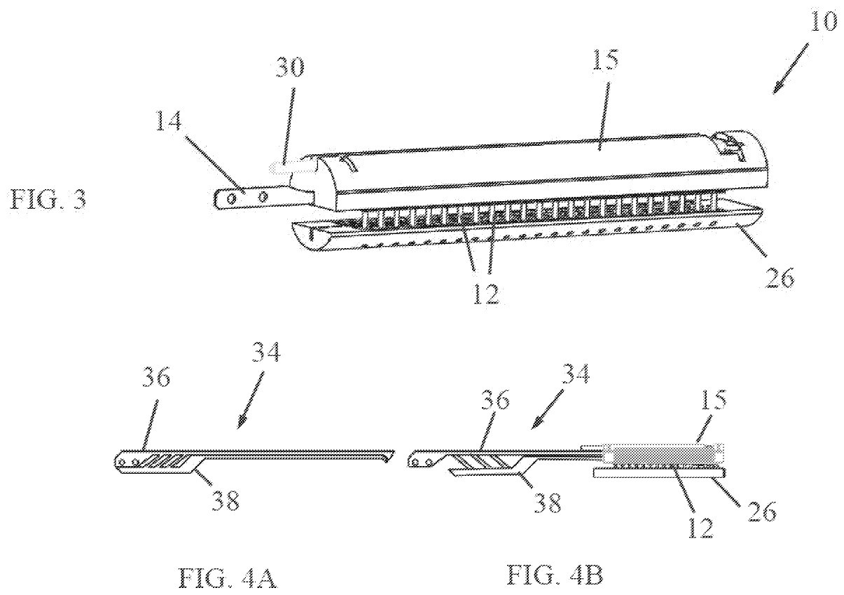

Reference is now made to FIGS. 1-3, which illustrate a surgical tacker assembly 10, constructed and operative in accordance with a non-limiting embodiment of the present invention.

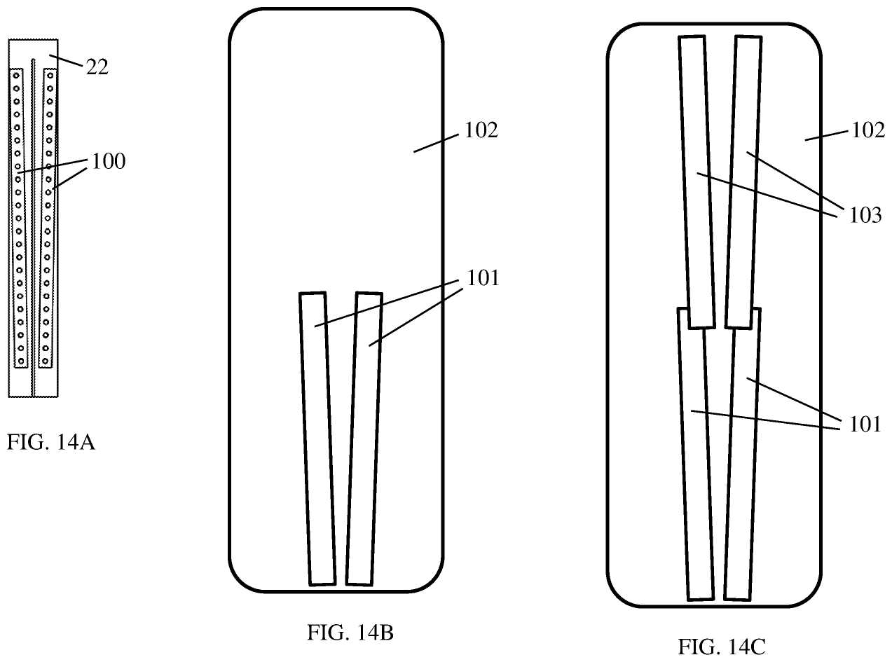

The surgical tacker assembly 10 may include one or more arrays of tacks (also referred to as fasteners) 12, such as one, two or six or any other number of rows of tacks 12. A cutting element 14 may be disposed between the arrays of tacks 12. In the illustrated embodiment of FIGS. 1-3, cutting element 14 is a blade; further below another cutting element will be described. If more than one row of tacks is used, the rows may be parallel or alternatively may not be parallel to each other, or any combination thereof. An example of non-parallel tack lines (rows) is described below with reference to FIGS. 14A-14C.

The arrays of fasteners 12 are not necessarily linear, but can also be circular, elliptical or any type of curved array.

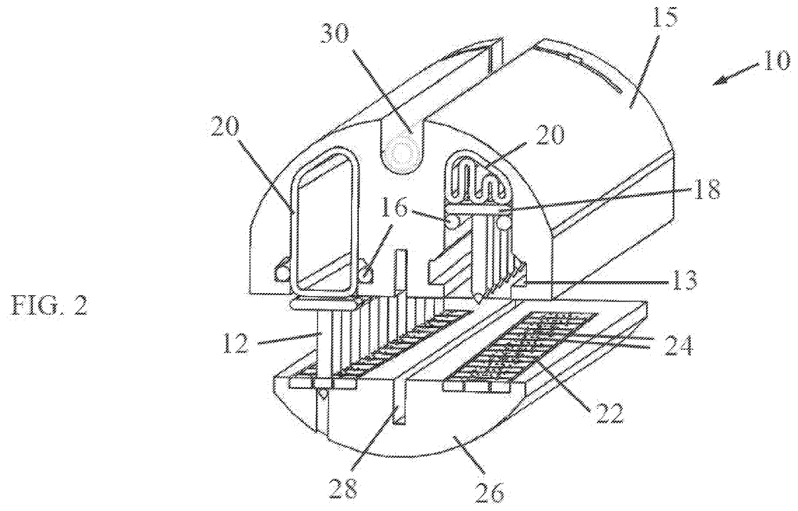

The left side of FIG. 2 shows the tacks 12 in the deployed position and the right side of FIG. 2 shows the tacks 12 in the stowed position.

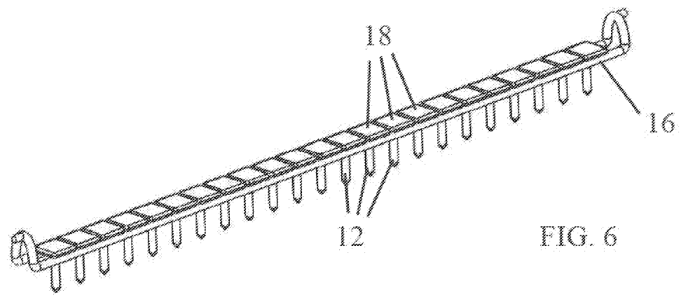

In the stowed position, the tacks 12 are supported by a flexible support member 16 (also shown in FIG. 6). In one non-limiting embodiment, flexible support member 16 includes two parallel flexible wires that can be flexed away from each other by a force and upon removal of the force the wires flex back to being parallel to each other. It is noted that the flexible support member 16 can be of any shape and size; the term “wire” encompasses wires, beams, rods and other members, and the terms are used interchangeably throughout. Each tack 12 may have a head portion 18 that rests upon the flexible wires while they are parallel. The “head portion” or simply “head” of the fastener is defined as the portion opposite to the sharp portion of the fastener. For example, for a tack, the head is generally collinear with the sharp point of the tack; for a staple, the head portion includes any or all of the part of the staple opposite the sharp ends of the staple. The head portion may be wider than the rest of the fastener (leg or legs of the fastener), but alternatively may be the same width. Without limitation, the wires may be parallel at all times from the stowed position to the deployed position. The wires may be made, without limitation, from nitinol or stainless steel or polymeric material.

The parallel wires upon which support the head portions 18 of the tacks 12, may be part of a pre-loaded spring. The spring may be assembled compressed, which means it constantly tries to relax by increasing the distance between the two wires. As will be described below, actuators 20 advance the tacks towards receivers 22. As the actuators advance the tacks toward the respective receivers, the wires spread apart and eventually reach longitudinal slots 13 (FIG. 2) in the upper housing 15 of the device. Entrance of the preloaded wires into the slots clears the way for the tacks to complete deployment.

Alternatively or additionally, as shown in FIG. 1, the wires of the flexible support member 16 may be urged by a biasing member 19, such as leaf springs. “Spring” and “biasing member” are used interchangeably throughout.

A fluid actuator 20 is aligned with tacks 12, such as being disposed against head portions 18 of tacks 12. Each array of tacks 12 has its own dedicated fluid actuator 20. Alternatively, there may be more than one fluid actuator for each array of tacks, so that only one or only some of the tacks may be actuated by a single fluid actuator. Without limitation, each fluid actuator 20 includes an elongate balloon. The left side of FIG. 1 shows the balloon in the stowed position (in which the balloon is deflated) and the right side of FIG. 1 shows the balloon in the deployed position (in which the balloon is inflated).

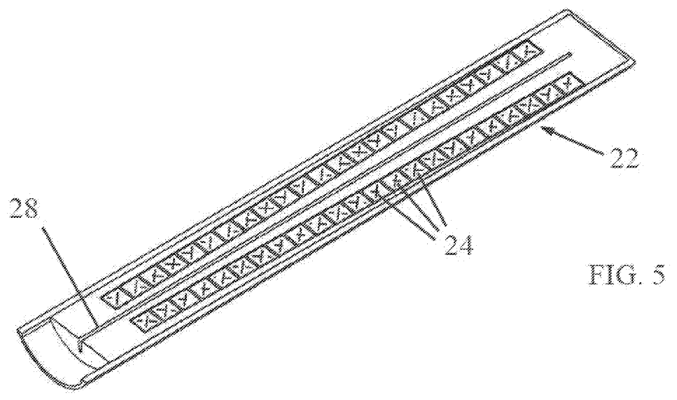

A receiver member (also called anvil) 22, also shown in FIG. 5, is formed with apertures 24 that are aligned with each of the tacks 12. The receiver member 22 is disposed in an anvil jaw (lower jaw) 26 of the surgical tacker assembly 10, as seen in FIGS. 1-3. Anvil jaw 26 may be formed with a slot 28 for the cutting element 14 to pass through.

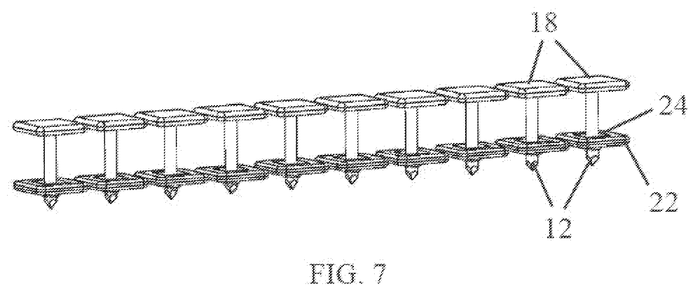

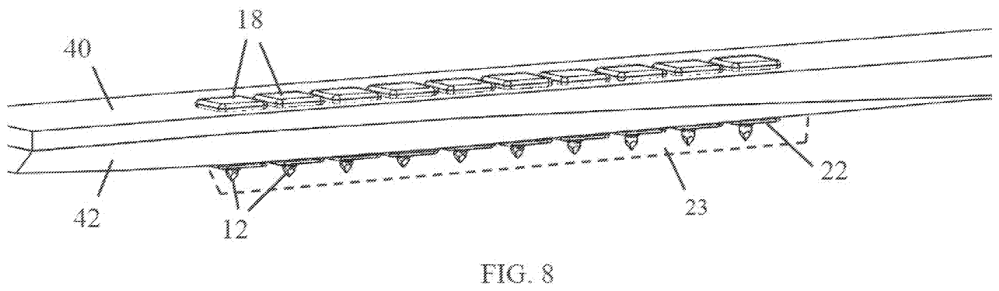

In use, tissues 40 and 42 that are to be tacked (not shown in FIGS. 1-3 but shown in FIG. 8) are placed between the pointed ends of tacks 12 and receiver member 22. Inflating fluid actuator 20 causes fluid actuator 20 (balloon 20) to expand and apply a pushing force on the tacks 12 (downwards in the sense of FIG. 1). Since the wires of flexible support member 16 are rounded (they could be alternatively non-round and formed with chamfers), the force of fluid actuator 20 pushes the heads of the tacks so the heads spread apart the wires (overcoming the urging force of biasing device 19), and the force pushes the tacks past the flexible support member 16. The force drives the pointed ends of tacks 12 through the tissues and through apertures 24 of receiver member 22, which are aligned with each of the tacks 12. FIG. 7 shows the tacks 12 that have passed through apertures 24; FIG. 8 shows the tissues 40 and 42 tacked by the tacks 12.

The cutting element 14 may also be propelled by fluid force (or alternatively by other forces, such as mechanical, electrical, etc.), such as simultaneously with firing of the tacks 12 (alternatively there may be a delay between actuation of the cutting element 14 and the tacks 12). For example, fluid actuator 20 may include another portion 30 that can abut against cutting element 14 to propel cutting element through slot 28. Alternatively, portion 30 may be a separate fluid actuator. The fluid actuator or actuators may be connected to a source of pressurized fluid (e.g., air or water), not shown in FIGS. 1-3.

The operation of cutting element 14 may be coordinated with deployment of the fasteners 12. Fasteners 12 may be deployed simultaneously as a group, where the entire fastener cluster is acted upon by fluid actuator 20 simultaneously. Another way for deploying the fasteners 12 is sequentially (one by one or in pairs), until all fasteners 12 are deployed. The cutting element 14 may cut only after the tissue in its vicinity has been sufficiently stabilized. When the tissue is restrained, cutting is possible. Clamped tissue restraining is highest just after fastener deployment and prior to jaw release. This is the time window to operate the cutting element 14.

In one non-limiting operation of cutting element 14, the blade travel is from the proximal to distal direction (e.g., the blade is pushed for cutting). The cutting operation begins only after all fasteners 12 have been deployed simultaneously as a group.

In another non-limiting operation of cutting element 14, the blade travel is from the distal to proximal direction (e.g., the blade is pulled for cutting). The cutting operation begins only after all fasteners 12 have been deployed simultaneously as a group.

Alternatively, the fasteners 12 may be deployed sequentially. The blade advancement (whether proximally or distally) may be simultaneous with fastener deployment or slightly delayed after fastener deployment, as the surrounding tissue gets restrained.

The assembly 10 may be introduced through a tube (not shown), which without limitation can be 5, 8, 10, 12 or 15 mm in diameter. Alternatively, assembly 10 may be housed in the tube. The assembly 10 may be coupled to an articulating shaft of a tacker (not shown).

Reference is now made to FIGS. 4A and 4B, which illustrate another cutter 34 that can be used in the tacker assembly of the invention. In this embodiment, cutter 34 includes first and second blade assemblies 36 and 38 that are movable with respect to each other. Movement of first and second blade assemblies 36 and 38 with respect to each other slices or otherwise cuts the tissue.

It is noted that the closed-jaw clearance of the surgical tacker assembly of the invention is in a suitable range for fastener deployment, taking into account such factors as tissue thickness, tissue condition, and others. In the present invention, whether using one-piece or two-piece fasteners, the range of closed-jaw clearance may include more than one clearance. For example, there may be a preset jaw closure of a relatively wider gap for stabilizing the tissue interface, and then a narrower clearance at deployment of the fasteners until the required (which may be preset) tissue clamping pressure is achieved. This provides several advantages. First, due to the first, wider clearance before fastener deployment, the clamped tissue interface is not over-stressed. Second, the final clamping pressure along the closure-line is very uniform. Third, the clamped tissue integrity is more likely to be maintained, resulting in a structurally sound interface, which is less prone to bleeding and leaking.

At least one of the jaws may be visually marked to aid the operator to quickly assess clamp-line length.

Each deployed fastener is atraumatic to neighboring tissue. The sharp tip of the fastener 12 that protrudes beyond the receiver member 22 may be protected by a protector 23 (shown in broken lines in FIG. 8) to prevent unwanted piercing of surrounding tissues. For example, protector 23 may be a thin foam layer of sufficient thickness attached to the outer side of the receiver member 22, such that the fastener tip is located in the protective foam layer 23. In another example, protector 23 may be an integral tang bent over the exit area of the receiver member 22, such that when the fastener tip protrudes out, it is capped by the tang and shielded from any adjacent tissue.

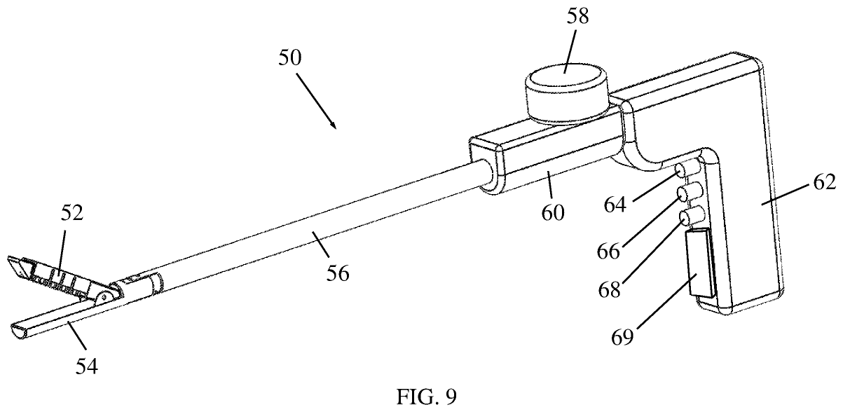

Reference is now made to FIG. 9, which illustrates a surgical tacker instrument 50, which uses the surgical tacker assembly of any of the embodiments shown herein.

The surgical tacker instrument 50 includes a cartridge jaw 52 for receiving the cartridge (any of the cartridges described herein) and an anvil jaw 54 for receiving the anvil (any of the anvils described herein). The jaws are coupled to a distal portion of a shaft 56. An articulation control 58 is operatively coupled to the jaws and shaft to articulate the jaws in azimuth (i.e., rotation perpendicular to the longitudinal axis of shaft 56). A rotation control 60 is operatively coupled to the jaws and shaft to rotate the shaft 56 (and with it the jaws 52 and 54) in yaw (i.e., rotation about the longitudinal axis of shaft 56). The surgical tacker instrument 50 includes a handle 62 and operational controls (buttons or handles), such as a cutting element control 64, a tack firing (ejector) control 66, a clamping pressure control 68, and a jaw opening and closing control 69 (such as a trigger or any other convenient control element).

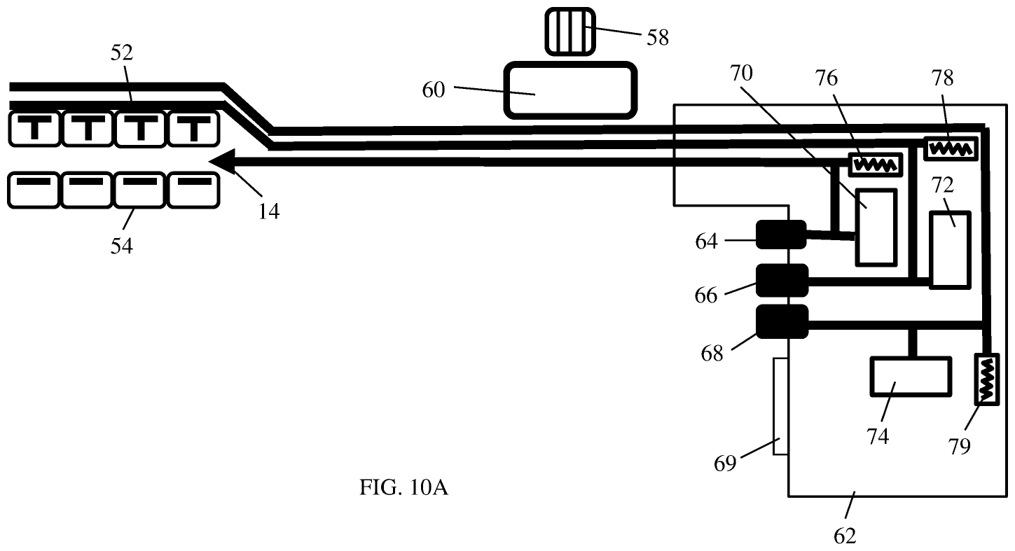

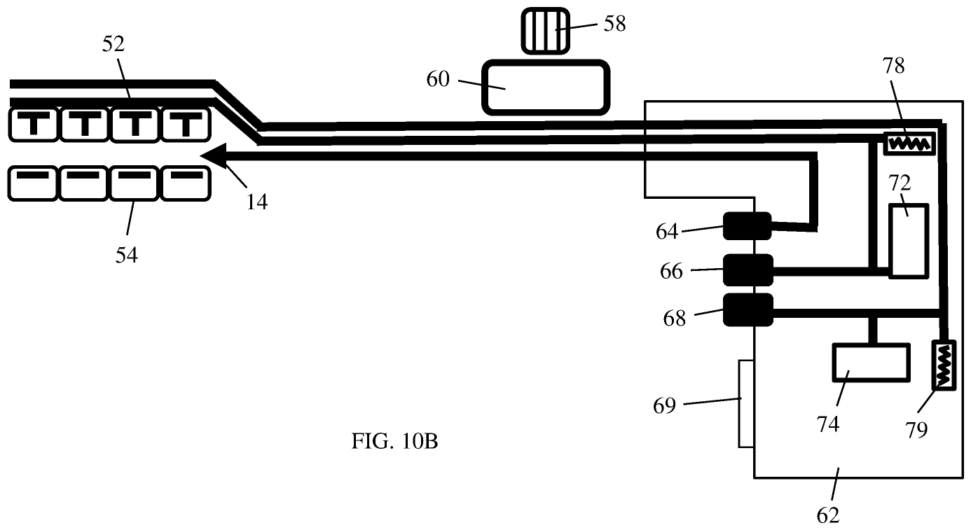

Reference is now made to FIG. 10A. In this embodiment, cutting element control 64, tack firing control 66, and clamping pressure control 68 are all fluid-operated and are coupled to fluid reservoirs 70, 72 and 74, and pressure sensors/indicators 76, 78 and 79, respectively.

Accordingly, in the embodiment of FIG. 10A, there are three independent fluid (hydraulic or pneumatic) lines that control the clamp pressure, the tack firing and the cutting; there are three independent non-fluid actuation controls for controlling opening/closing of the jaws, jaw articulation and shaft rotation.

In the embodiment of FIG. 10B, cutting element control 64 is not fluid-operated and instead may be operated in any of the other ways described above. The tack firing control 66 and clamping pressure control 68 are fluid-operated and are coupled to fluid reservoirs 72 and 74, and pressure sensors/indicators 78 and 79, respectively.

Accordingly, in the embodiment of FIG. 10B, there are two independent fluid (hydraulic or pneumatic) lines that control the clamp pressure and the tack firing; there are four independent non-fluid actuation controls for controlling cutting, opening/closing of the jaws, jaw articulation and shaft rotation.

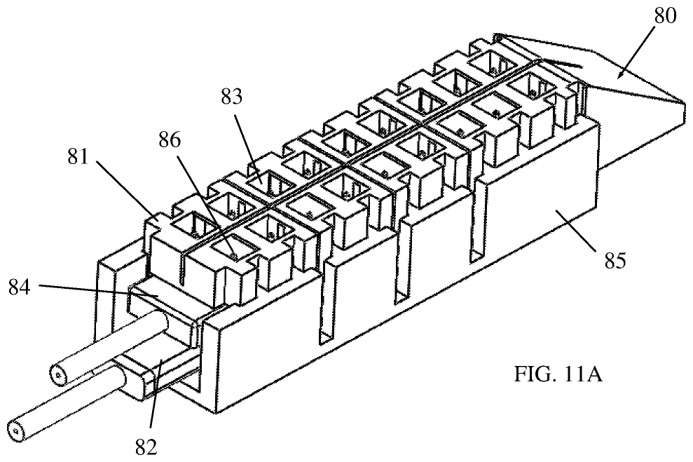

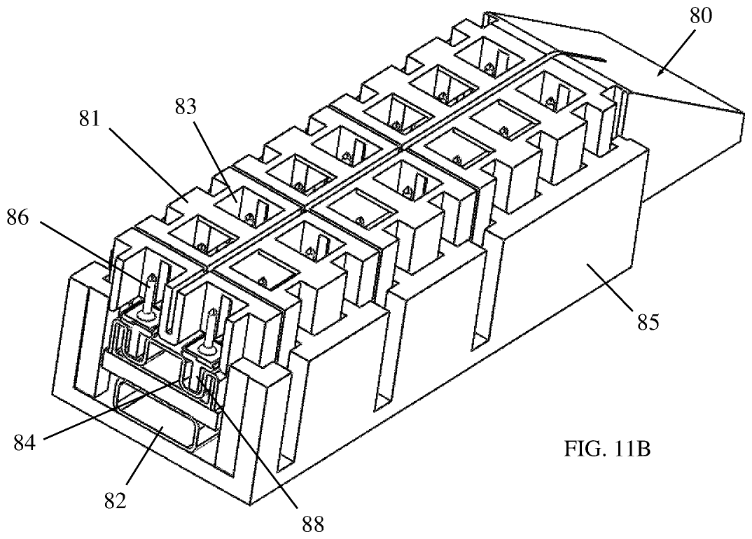

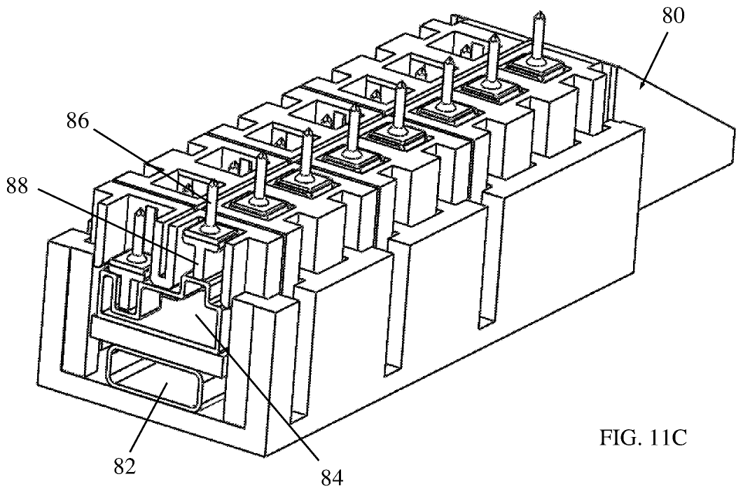

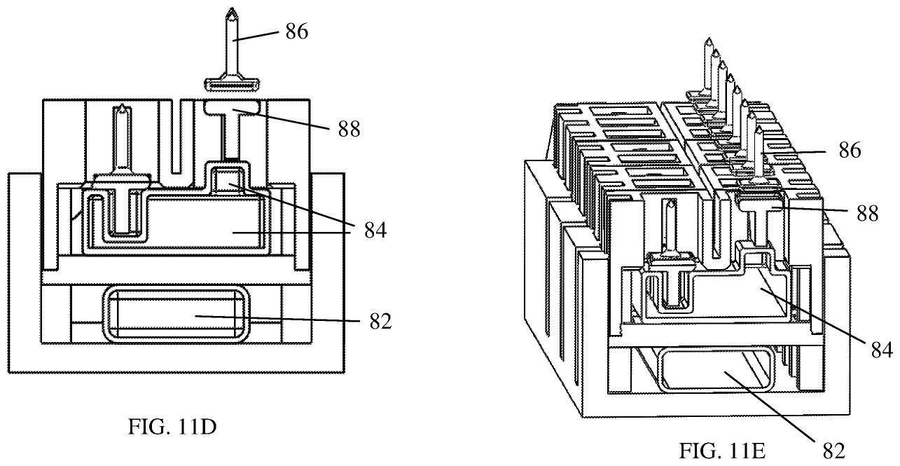

Reference is now made to FIGS. 11A-11C, which illustrate a tack cartridge 80 of the surgical tacker assembly for use with the instrument of FIG. 9. The tack cartridge 80 may include a clamp balloon 82 that provides a distributed force to clamp tissue before tacking and cutting, and a tack balloon 84 that provides a force to fire (eject) the tacks from the cartridge. (The terms “balloon” and “fluid actuator” are used interchangeably.) Tacks 86 (FIG. 11B) are arranged in rows of a cartridge block 81 formed with openings 83, one opening for each tack. The cartridge block 81 is mounted in a cartridge base 85 of cartridge 80. Each tack 86 is mounted on a tack driver 88 which can be moved outwards by the expansion of tack balloon 84 and retracted inwards to its original position by contraction of tack balloon 84.

It is noted that the tack driver 88 and tack balloon 84 may be arranged to eject not just one row of tacks 86 but both rows of tacks 86.

FIG. 11B shows deployment (expansion) of the clamp balloon 82. FIGS. 11C, 11D and 11E show deployment (expansion) of the tack balloon 84 (on the right side row, with the left tack balloon still contracted and not yet deployed.

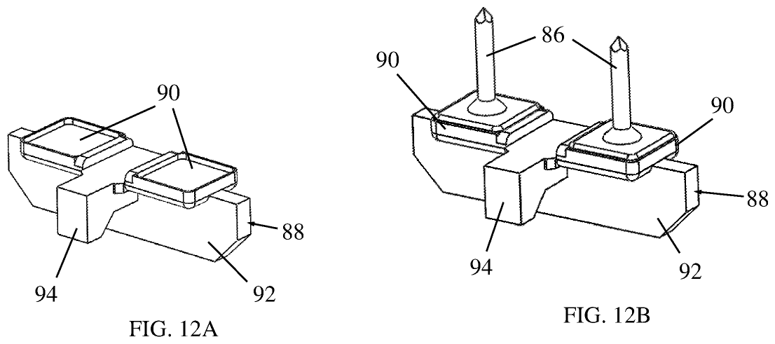

Reference is now made to FIGS. 12A and 12B, which illustrate tack holders (also called support members) 90 for holding the tacks 86. The tack holder 90 may have a socket shaped to complement the shape of the tack head, such as but not limited to, a square shape. The tack holder 90 is coupled to the tack driver 88. The head of tack 86 may be held in place in the tack holder 90, such as but not limited to, an interference fit, a snug fit, a snap fit, a gel or adhesive (strong enough to hold the tack in place until the tack is driven into tissue, and then yields to the force holding the tack in the tissue so that the head of the tack comes out of the tack holder and the tack remains in the tissue) or other means or combination of means. In the case of an interference, snug, or snap fit, the tack holder may be somewhat resilient to yield to the force of the fit, and thus serves as a kind of flexible support member. The force of the expansion of the tack balloon on the tack driver 88 (or directly on the tack 86, or against the tack stabilized in support member 16) propels the tack 86 to be inserted in the tissue. When the jaws are opened, the tack holder 90 is pulled away by the jaws opening and the contraction of the tack balloon, leaving the tack 86 in the tissue.

Accordingly, in use, tissue to be tacked is placed between cartridge jaw 52 and anvil jaw 54. The jaw opening and closing control 69 is used to close cartridge jaw 52 towards anvil jaw 54 to sandwich the tissue there between. The clamping pressure control 68 is then used to expand the clamp balloon 82 in order to clamp the tissue with controlled pressure. The tack firing control 66 is then used to expand tack balloon 84 to drive the tacks 86 into the tissue. The cutting element control 64 is then used to actuate the cutting element to cut the tissue between the rows of tacks. The clamping pressure control 68 is then used to contract the clamp balloon 82 to relieve the clamping pressure from the tissue. The jaw opening and closing control 69 is then used to open cartridge jaw 52 away from anvil jaw 54 to release the tacked tissues.

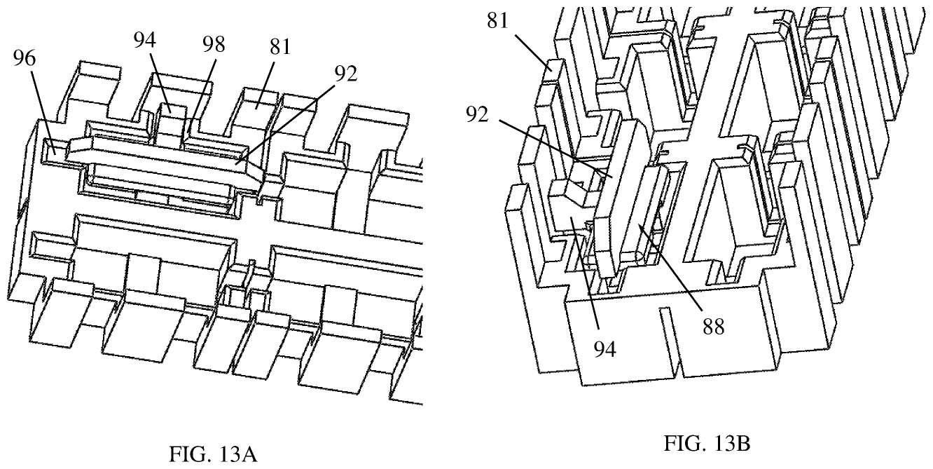

As seen in FIGS. 12A, 12B, 13A and 13B, each tack holder 90 may include one or more guides, such as first and second guides 92 and 94, which help guide the tack 86 (and tack holder 90) to move forward axially, and stabilize the tack holders 90 in different angular orientations, such as roll, yaw and pitch, so that the tack or tack holder does not turn or wobble. FIGS. 13A and 13B show the first and second guides 92 and 94 received in slots, grooves or channels 96 and 98, respectively, formed in cartridge block 81.

As mentioned previously, if more than one row of tacks is used, the rows may be alternatively not parallel to each other. An example of non-parallel tack rows is described now with reference to FIGS. 14A-14C. In FIG. 14A, two non-parallel tack rows 100 are formed in any of the receiver members of the invention, such as receiver member 22. The non-parallel rows 100 enable overlapping of two different tack lines to reduce leakage. It is noted that rows 100 may be straight or alternatively may be curved in any curved shape depending on the application. This is seen in FIG. 14B, which shows a first tack line 101 formed in tissue 102, followed by a subsequent tack line 103 formed in tissue 102, as seen in FIG. 14C. The overlapping of the tack lines 101 and 103 may provide an improved securing of tissue in a smaller area.

Accordingly, in all embodiments of the invention, a head portion of the surgical fastener is held by a fastener head holding member. In the embodiment of FIGS. 1-3, the fastener head holding member is the flexible support member 16. In the embodiment of FIGS. 11A-11E, the fastener head holding member is the tack holder 90. The fluid actuator provides a fluid force to propel a sharp end of the fastener forward. The fluid actuator provides a fluid force to free the head portion of the surgical fastener from the fastener head holding member. In the embodiment of FIGS. 1-3, the fluid force that frees the head portion of the fastener from the fastener head holding member is the fluid force that pushes the tack heads to spread apart the wires of the flexible support member 16 (which may also overcome the urging force of biasing device 19). In this case, this fluid force is the same fluid force that propels the sharp end of the fastener forward. In the embodiment of FIGS. 11A-11E, the fluid force that frees the head portion of the fastener from the fastener head holding member is the fluid force that contracts the tack balloon 84 so that the tack holder 90 is pulled away from the tack head, leaving the tack in the tissue. In this case, this fluid force is different from the fluid force that propels the sharp end of the fastener forward.

What is claimed is:

1. A surgical fastener assembly comprising:
one or more arrays of fasteners, each of said fasteners comprising a head portion opposite to a sharp portion;
a fastener head holding member, each of said head portions of said fasteners being held by said fastener head holding member;
a receiver member aligned with each of said one or more arrays of fasteners; and
a fluid actuator for each of said one or more arrays of fasteners, each of said fluid actuators being contractible and expandable, and wherein each of said fluid actuators is configured to provide a force against its corresponding array of fasteners to propel said sharp portions of said fasteners towards said receiver member and also configured to provide a force to free said head portions from said fastener head holding member.

2. The surgical fastener assembly according to claim 1, wherein said fastener head holding member is a flexible support member.

3. The surgical fastener assembly according to claim 1, wherein said fastener head holding member comprises a socket shaped to complement a shape of said head portion.

4. The surgical fastener assembly according to claim 1, wherein each of said fluid actuators when expanded provides said force to propel said sharp portions of said fasteners towards said receiver member and said force to free said head portions from said fastener head holding member.

5. The surgical fastener assembly according to claim 1, wherein each of said fluid actuators when expanded provides said force to propel said sharp portions of said fasteners towards said receiver member and when contracted provides said force to free said head portions from said fastener head holding member.

6. The surgical fastener assembly according to claim 1, further comprising a cutting element disposed adjacent said one or more arrays of fasteners.

7. The surgical fastener assembly according to claim 6, wherein said fluid actuator is coupled to said cutting element.

8. The surgical fastener assembly according to claim 6, wherein said receiver member is disposed in an anvil jaw that is formed with a slot for said cutting element to pass through.

9. The surgical fastener assembly according to claim 1, wherein said fastener head holding member comprises two parallel flexible wires.

10. The surgical fastener assembly according to claim 2, further comprising a biasing member configured to apply an urging force on said flexible support member.

11. The surgical fastener assembly according to claim 1, wherein said cutting element comprises first and second blade assemblies that are movable with respect to each other.

12. The surgical fastener assembly according to claim 1, wherein said fluid actuator comprises an inflatable and deflatable balloon.

13. The surgical fastener assembly according to claim 1, further comprising a clamp fluid actuator coupled to said one or more arrays of fasteners and operative to move said one or more arrays of fasteners.

14. The surgical fastener assembly according to claim 1, wherein said support member comprises a holder in which each of said fasteners is received with an interference fit, a snug fit, a snap fit, a gel or an adhesive.

15. The surgical fastener assembly according to claim 14, wherein said holder comprises one or more guides.

16. A method of fastening tissues comprising:
placing a tissue between said fasteners and said receiver member of the surgical fastener assembly of claim 1; and
expanding said fluid actuator to apply a force on said fasteners to drive the sharp portions of said fasteners into said tissue.
